(19) United States
(12) Patent Application Publication
(10) Pub. No.: US 2025/0002571 A1
(43) Pub. Date: Jan. 2, 2025

(54) Camelid/Llama Nanobodies to Target WNT16B as a Chemotherapy Option for Cancer Treatment

(71) Applicant: Applied Bio Research Inc., Windsor (CA)

(72) Inventors: Maryam Jassim, Windsor (CA); Sabah A A Jassim, Windsor (CA); Hassan El-Cheikh, Windsor (CA); John W. Hudson, Tecumseh (CA)

(21) Appl. No.: 18/760,196

(22) Filed: Jul. 1, 2024

Related U.S. Application Data
(60) Provisional application No. 63/511,547, filed on Jun. 30, 2023.

(51) Int. Cl. C07K 16/22; A61K 45/06; A61P 35/00; G01N 33/574

(52) U.S. Cl. CPC C07K 16/22; A61K 45/06; A61P 35/00; G01N 33/5748; C07K 2317/22; C07K 2317/73

(57) ABSTRACT

A method for targeting cancer cells is described. The method uses immunizing a dromedary camel or llama with a WNT16B molecule to generate anti-WNT16B antibodies, then preparing a set of anti-WNT16B IgG blocking antibodies from the camel or llama. Cancer cells are targeted with the blocking anti-WNT16B IgG antibodies, which can be used for more effective chemotherapy and radiotherapy. The anti-WNT16B IgG antibodies can also be used in vitro or in vivo to detect cancer cells.

This application claims priority to U.S. provisional application 63/511,547, filed on Jun. 30, 2023, the contents of which are hereby incorporated by reference.

### BACKGROUND

Long considered the most effective cancer-fighting treatment, chemotherapy may make cancer worse, according to a study by Sun et al. (2012), which reports that healthy cells damaged by chemotherapy secreted increased amounts of a protein called WNT16B that boosts cancer cell survival. DNA damage from cytotoxic therapies causes healthy stromal cells within the tumor neighborhood to secrete proteins that can influence the tumor's response to therapy. The authors showed that benign primary prostate fibroblasts upregulated the expression of WNT family member wingless-type MMTV integration site family member 16B (WNT16B) by up to 64-fold as a result of the genotoxic drugs or radiation therapy. This upregulation of WNT16B in the tumor microenvironment promoted cancer therapy resistance and led to increased tumor growth and invasiveness. Previous studies have shown that WNT16B secretion normally regulates the tumor suppressor p53 in response to DNA damage and is critical for the onset of senescence, showcasing a linkage between WNT16B expression and cell survival (Binet R. et al., 2009). In cancer cells, the overexpression of WNT16B as a response to DNA damage from chemotherapy treatment was instead found to induce epithelial-to-mesenchymal transition and incur resistance towards chemotherapy, thereby increasing the metastatic potential and survival of the cancer cells respectively (Sun Y et al., 2012). Such findings outline a mechanism by which cytotoxic therapies given in cyclical doses may augment later treatment resistance.

### SUMMARY

An object of the present disclosure is to provide methods and compositions for the treatment of cancer using camelid/llama antibodies targeted against WNT16B in association with chemotherapy.

Thus, by one aspect of the present invention, a method is provided for targeting of cancer cells by generating anti-WNT16B immune cells.

By another aspect of the present invention, a method to detect cancer cells is provided comprising anti-WNT16B immune cells, selecting a set of a set of anti-WNT16B IgG antibodies and using the antibodies to detect cancer cells.

### DETAILED DESCRIPTION

Camelid IgG is promising as a potential therapeutic since they are robust and yet small enough for rapid elimination from the body and easy to produce at a relatively low cost (Jassim unpublished data). The present disclosure focuses on immunizing a dromedary camel to create antibodies unique to WNT16B. Next, all unnecessary elements of the camel antibodies are removed to harvest elite nanobodies that specifically target WNT16B, which are cloned in a phage display vector or E. coli. The phage clones that produce the specific nanobodies that recognize and block WNT16B protein are selected. These nanobodies allow a rapid diagnostic for the WNT16B protein and rapid detection of regrowth and resistance to further chemotherapy.

This technique contributes to targeting cancer cells with more effective chemotherapy and also identifies other discrete cancer growth. This is a highly promising approach for improving the efficacy of chemotherapy by targeting tumor cells directly while minimizing damage to healthy cells or the spread of cancer to other locations in the body.

### Scientific Evidence and International Works on Camels

The advantages of camel antibodies over conventional antibodies have been reported (Jassim and Naji 2001), and it was found that Camelus dromedarius has a particularly effective immune system, unique among mammalians. Subclasses IgG2 and IgG3 (which occur naturally in Camelidae) consist of only two heavy chains connected by several disulphide bonds in the hinge region. The light chains are also missing entirely (Jassim and Naji 2001).

A major setback to the development of immunotherapy up until now is the size and complexity of antibodies; past a critical point, the antibody is unable to reach its target. The camel heavy-chain antibodies present advantages over common antibodies in the design, production, and application of clinically valuable compounds (Transue et al., 1998; Jassim and Naji 2001).

It was subsequently found that the camelid variable domain (VHH) of these heavy-chain antibodies have long complementarities determining region (CDR3) loop, which compensates for the absence of the antigen-binding loops of the variable light chains (VL) (Desmyter et al., 1996; Muyldermans et al., 2001). This could provide a possible route to eradicate pathogens and prevent immunosuppression diseases. Furthermore, camel IgG has a full neutralizing capacity against tetanus toxin due to its capability to intrude into the enzyme's active structure (Riechmann and Muyldermans, 1999) with a potent and selective inhibitor of the hepatitis C virus' serine protease enzyme (Martin et al., 1997).

Conventional antibodies do not appear to interact with the tetanus toxin enzyme's active site. It thus appears that VHH domains are better suited to be enzyme inhibitors than human or mouse antibody fragments (Riechmann and Muyldermans, 1999). Both immunized dromedary sera and heavy chain antibody subclasses can neutralize the toxicity of Aah toxins in mice (Meddeb-Mouelhi et al. 2003).

The study of Jassim et al. (2011) described a method of producing immunoglobulin that specifically binds to cancer cells by immunizing a camelid with tumor-associated antigen protein, purified from cancer cells by acid elution. The tumor-associated antigen-specific immunoglobulin is then extracted from camelid body fluid. The resulting cancer cell-specific camelid immunoglobulins or immunotoxins are then used to treat or prevent cancer.

The nanobody technology was developed following the discovery that Camelidae (camels and llamas) possess fully functional antibodies that lack light chains. Importantly, the cloned and isolated VHH domain is a perfectly stable polypeptide harboring the full antigen-binding capacity of the original heavy-chain antibody. These newly discovered VHH domains, with their unique structural and functional properties, form the basis of a new generation of therapeutic antibodies named VHH domain (˜15 kDa) Nanobody (Deffar et al. 2009).

Cloning the repertoire of antigen-binding fragments from an immunized camel into a phage display vector and selection of antigen-specific clones by panning has become a routine method for selecting antigen-specific molecules in the past decade (Revets et al., 2005).

### Previous Projects, Outcomes and Market Potential

Many companies are searching for ways to use small antibodies. The U.K. Company Haptogen, which was bought by Wyeth in 2007, used antibodies from sharks, another animal known to produce small antibodies. Domantis, a U.S. company bought by Glaxo SmithKline PLC in 2006, snips off part of the human antibody using laboratory techniques that produce something like a llama antibody.

Despite their promise, no one knows whether drugs using small antibodies will work in humans. However, Jassim and his team (2011) reported none of the 100 mice that were transplanted with Ichikawa cells and treated with the specific anti-T-cell leukemia purified camelid TAA IgGs-RA conjugates developed any tumor, and they were indistinguishable from healthy normal nude mice. These results strongly suggest that purified camelid TAA IgGs-RA conjugates have formed the basis for proceeding with novel camelid immunotherapy to target and treat cancer with clinical trials in humans.

### EXAMPLE—BLOCKING THE ACTIVITY OF WNT16B WITH CAMELID IGG

### Methods

### 1. Tissue Culture

The following human cancer cell lines were cultured and were used for testing the efficacy of the WNT16B Camelid IgG in blocking the activity of WNT16B in vitro.

HeLa cells, which are epithelial cells from cervical adenocarcinoma from a 31-year-old patient, were obtained from ATCC. The patient underwent radiation therapy and radium treatment for cervical adenocarcinoma treatment (John Hopkins Medicine). The HeLa cells were grown in DMEM complete growth medium (Sigma-Aldrich, Canada) supplemented with 15% Fetal Bovine Serum and 5% sodium Pyruvate. They were incubated at 37° C. with 5% CO₂. The HeLa cells underwent aa media renewal 2 to 3 times per week and were subcultured at a ratio of 1:2 or 1:6 when the cells reached 80% cell confluency.

HepG2 cells are hepatocellular carcinoma with epithelial-like morphology that were isolated from a 15-year-old white male youth with liver cancer, and were obtained from ATCC. The patient's treatment history was not well documented. The HepG2 cells were grown in DMEM complete growth medium from Sigma-Aldrich supplemented with 15% Fetal Bovine Serum and 5% sodium Pyruvate. They were incubated at 37° C. with 5% CO₂. The HepG2 cells underwent aa media renewal 2 times per week and were subcultured at a ratio of 1:4 or 1:6 when the cells reached 80% cell confluency. SAOS-2 cells are derived from an 11-year-old white female osteosarcoma patient. The patient was treated with the following therapies: RTG, methotrexate, adriamycin, vincristine, cytoxan, and aramycin-C (ATCC). The SAOS-2 cells have an epithelial morphology and were obtained from ATCC. The SAOS-2 cells were grown in McCoy's 5a Medium from Sigma-Aldrich supplemented with 15% Fetal Bovine Serum. They were incubated at 37° C. with 5% CO₂ and underwent a media renewal 1 to 2 times a week with a subcultivation ratio of 1:2 or 1:4 at 80% cell confluency.

MCF7 cells are epithelial cells from a breast/mammary gland adenocarcinoma from a 69-year-old white female who underwent radiation therapy, hormone therapy, and multiple chemotherapeutic agents. (Soule et al. 1973.) The MCF7 cells were obtained from ATCC and were grown in DMEM complete growth medium from Sigma-Aldrich supplemented with 15% Fetal Bovine Serum and 5% sodium Pyruvate. They were incubated at 37° C. with 5% CO₂. The MCF7 cells underwent a media renewal 2-3 times per week and were subcultured at a ratio of 1:3 or 1:6 when the cells reached 80% cell confluency.

U-2OS cells with an epithelial morphology were derived from a 15-year-old white female osteosarcoma patient with a moderately differentiated sarcoma of the tibia. The patient's treatment history was not well documented. The U-2OS cells were grown in McCoy's 5a Medium (Sigma-Aldrich) supplemented with 15% Fetal Bovine Serum. They were incubated at 37° C. with 5% CO₂ and underwent a media renewal 2 to 3 times a week with a subcultivation ratio of 1:3 or 1:6 at 80% cell confluency.

### 2. Protein Extraction of Cancer Cell Lines

Proteins were extracted from the following cell lines at 80% cell confluency: HeLa, HepG2, MCF7, SAOS-2, and U-2OS. The growth media of the aforementioned cancer cell lines was removed, and the cancer cell lines were washed twice with 10 mL of 1% phosphate buffer saline (PBS) solution at pH 7.4. The PBS solution was removed, and 1000 μL of lysis buffer supplemented with 50 μL of sodium fluoride (NaF) and 13.5 μL of phenylmethylsulfonyl fluoride (PMSF) was added to each tissue culture plate containing each of the aforementioned cancer cell lines. The cells were scraped off the tissue culture plate with a cell scraper, and the 1000 μL of the scraped cells were put into an Eppendorf tube and incubated on ice for 5 minutes. The lysed cells in the Eppendorf tube were then centrifuged for 20 minutes at 4° C. at 13000 rpm. The supernatant containing the cell proteins was removed and put into a new Eppendorf tube and stored in −20° C.

A Bradford assay of the proteins from the lysed cells was carried out to ensure equal loading of protein onto a 10% acrylamide gel. 20 μL of the protein from the cells were mixed with 4 μL of 6× loading dye, and incubated at 95° C. for 5 min to denature the proteins and spun down. Then 20 μL of the denatured protein extracted from each of the aforementioned cancer cell lines was added to the wells of a 10% acrylamide gel along with 6 μL protein ladder.

### 3. Establishing WNT16B Expression in Cancer Cell Lines

The SDS-PAGE gel carrying the extracted proteins was run at 200V for 45 min. The gel was then transferred to a membrane activated by methanol using 1× transfer buffer through a dry-transfer method. The transfer system was run at 10V for 50 min, and after the transfer the membrane was allowed to dry completely. The membrane was then re-activated in methanol and washed 3 times on a shaker with 1×TBST, consisting of tris-buffered saline and Polysorbate 20. The membrane was then blocked in 5% skimmed milk mixed with 1×TBST and kept at 4° C. overnight. After the blocking procedure, the milk was removed and the membrane was washed 3 times with 1×TBST on a shaker and then probed by a 6 μL WNT16B monoclonal rabbit antibody in 8 mL of 5% skimmed milk mixed with 1×TBST for 1 hour at room temperature on a shaker. The membrane was again washed 3 times with 1×TBST on a shaker and probed with a secondary antibody, 2 μL of GAR-HRP (goat anti-rabbit-Horseradish peroxidase) in 8 mL of 5% skimmed milk mixed with 1×TBST for 1 hour at room temperature on a shaker. The membrane was once again washed with 3 times with 1×TBST on a shaker. The bands depicting the presence of WNT16B on the membrane were analyzed using chemiluminescence.

4. Protein Quantification from Western Blot Analyses

The western blot membranes that were probed with the WNT16B primary antibody from section 3 “Establishing WNT16B Expression in Cancer Cell Lines”, were incubated on a shaker at room temperature with a membrane stripping buffer for 10 min 2 times. The stripping buffer was then discarded and 1×PBS was added to the membrane and incubated on a shaker at room temperature for 10 min 2 times. The PBS was then discarded and the membrane was then incubated in 1×TBST on a shaker at room temperature for 5 min 2 times. The membrane was incubated in 5% milk mixed with 1×TBST overnight at 4° C. The membrane was probed with a primary antibody for the housekeeping gene GAPDH for 1 hour on a shaker in 5% milk mixed with 1×TBST at room temperature. The solution carrying the primary antibody was discarded and the membrane was then probed with a secondary antibody conjugated to a chemiluminescent tag and incubated at room temperature on a shaker. The membrane underwent chemiluminescent imaging.

The images of the membranes probed with WNT16B and GAPDH were analyzed through the Imagelab (Bio-Rad) software in order to quantify the amount of WNT16B present in the cancer cell lines by comparing its concentration to that of GAPDH.

5. Establishing WNT16B Secretion into the Media of Cancer Cell Lines

Since WNT16B is a secreted glycoprotein needed its for the intercellular signaling pathway known to drive cancer cell proliferation and metastasis, as well as cancer recurrence (Sun et al. 2012), a western blot was carried out in order to establish secretion of the WNT16B protein into the cell culture media of the aforementioned cancer cell lines. In order to optimize the western blot imaging, a molecular weight cut off filtration system was used. Cell culture media from the aforementioned cancer cell lines was collected into Falcon tubes and Eppendorf tubes when the cells grew up to 80% confluency. The 500 μL of the media from each cell line were filtered through centrifugal filters with a 50 kDa molecular weight cut off at 12,500 rpm at 4° C. for 25 minutes. The flow-through was collected and put into a separate Eppendorf tube. This process was repeated 2 more times, with the flow through being collected into the Eppendorf tube containing the first round of media flow through. The samples were kept stored at −20° C.

Next, a western blot was carried out using the same methods from section 3 on the cell culture media flow through above. Please refer to section 3 “Establishing WNT16B Expression in Cancer Cell Lines” for methods.

### 6. Establishing Expression of Downstream Targets of the WNT-β-Catenin Pathway

To analyze the full effect and efficacy the WNT16B Camelid IgG treatment has on blocking the activity of WNT16B, the levels of expression of the downstream targets of the WNT/β-catenin pathway were analyzed before and after treatment with the WNT16B camelid antibody. Proteins were extracted from HEK293, HeLa, HepG2, MCF7, SAOS-2, and U2-OS cells as per the methods highlighted in section 2 “Protein Extraction of Cancer Cell Lines”. A western blot analysis was carried out to detect the expression of the following downstream target proteins of the WNT-β-catenin signalling pathway in the aforementioned cancer cell lines: β-catenin, C-myc, Cyclin D1 (CCND1), AXIN2, LEF1, TCF, CD44, Survivin (BIRC5). (Clevers et al., 2012.) The western blot membranes were separately probed with antibodies that detected the downstream target proteins of the WNT-β-catenin pathway, known to play a role in cancer cell proliferation and cancer cell metastasis. The western blot analysis was carried out as per methods stated in section 3 “Establishing WNT16B Expression in Cancer Cell Lines”, however instead of using a primary antibody to probe for WNT16B, the primary antibodies used for each western blot analysis was specific to the respective downstream target protein(s) of the WNT-β-catenin pathway that were being analyzed.

Protein quantification for the levels of β-catenin, C-myc, Cyclin D1 (CCND1), AXIN2, LEF1, TCF, CD44, Survivin (BIRC5) from the western blot analysis was carried out using the same methods as per section 4 “Protein Quantification from Western Blot Analyses.”

The western blot analyses and protein quantification of β-catenin, C-myc, Cyclin D1 (CCND1), AXIN2, LEF1, TCF, CD44, Survivin (BIRC5) were carried out prior to cell culture treatment with the WNT16B camelid IgGs and repeated after treatment with the WNT16B camelid IgGs to analyze the effectiveness of the WNT16B camelid IgGs in blocking the intercellular signalling activity of WNT16B.

7. Camel Immunization with WNT16B Protein

A non-pregnant Dromedary camel with her male calf at the age of 5 years old and six months, respectively, were selected. A veterinarian was recruited to inject the 5-year-old Dromedary female camel with the WNTI6 protein. Each vial of WNT16B protein contained 125 μL of the WNT16B protein which was dissolved in phosphate buffered solution (PBS). The lyophilized WNT16B was stored at −20° C. A dosage of 15.5 μg was needed for each dosage which is the equivalent of 75 μL of lyophilized WNT16B. Therefore 75 μL of lyophilized WNT16B was added to 925 μL of PBS and transferred to a 21 G syringe. The Gauge was changed to be suitable for 1 M administration. The female camel was immunized with 75 μL of lyophilized WNT16B dissolved in 925 μL of PBS per dosage at 0, 7, 14, 21, 28, 35, 42, and 49 days (day 0 refers to the first day of immunization after the urine, milk, and blood samples were collected prior to immunization). A total of 8 doses were delivered. Sample collection was carried out at the specified time points (pre-dose at day 0, prior to the fifth dose at 28 days, and at 49 days). The last sample was collected one week after the last dose was administered, which depended on the camel's overall health. During the immunization period, the six-month-old male calf was allowed to nurse from the 5 year-old Dromedary female camel. Blood (Plasma), urine and milk samples were collected from the 5 year-old Dromedary female camel at the specified time points prior to the first immunization dose, prior to the fifth dose, and 7 days after the last dose. Furthermore, blood and urine samples were collected from the 6-month old male calf at the specified time points prior to the first immunization dose, prior to the fifth dose, and 7 days after the last dose. The collected blood samples from both the 5 year-old Dromedary female camel and 6-month-old male calf were transferred to EDTA.

Samples were stored in −80° C. until the time of quantification analysis and antibody purification.

### 8. Camel Whey Preparation Protocol

The milk samples were de-lipidized (skimmed) by centrifugation at 10,000 RPM for one hour at 0° C. Next the lipid layer was discarded and the skimmed milk was poured into a new container. De-caseination of the milk was carried out by adding Rennet enzyme (50 mg/L, Valiren Rennet & Ingredients, USA) after warming up the skimmed milk at 37° C. The mixture was stirred for 5 min and left for 25 min (without stirring). The skimmed milk samples was then centrifuged at 10,000 RPM, 0° C. for 30 minutes. Acid precipitation was used at pH 4.5 by the addition of 10% (v/v) acetic acid (1 M), and the skimmed milk was then incubated at 37° C. for one hour to enhance casein precipitation. The pH was then adjusted to 7.0 with 1 mL of NaOH (1 M) and re-centrifuged at 10,000 rpm for 30 min at 4° C. The centrifugation process was repeated if needed at 10,000 rpm at 0° C. for 30 min.

For sterilization, the whey was filtered using 0.45 μm cellulose membrane filter and then stored at 4° C. for further processing.

### 9. Camelid Antibody Quantification and Purification

Camelid IgG antibody detection and quantification in the blood/plasma (serum), milk/whey, and urine samples were carried out using an ELISA assay. The Camel immunoglobulins G (IgG1, IgG2 & IgG3) were purified from serum/plasma, milk/whey, and urine using protein G & A affinity chromatography. The following buffers and reagents were used for the camelid IgG purification:
Binding Buffer: 20 mM Sodium Phosphate Buffer (pH: 7.0).
Elution Buffer G1: 0.15 M NaCl, 0.58% Acetic Acid (pH: 3.5).
Elution Buffer G2: 0.1 M glycine Buffer (pH: 2.7).
Elution Buffer A1: 0.15 M NaCl, 0.58% Acetic Acid (pH: 4.5).
Neutralizing Buffer: 1 M Tris-HCI (pH: 9.0).
   Note: before use, filter all buffers using 0.45 μm syringe filter and then degas using the water-bath sonicator for 10-15 minutes at 25 C.
Protein A HiTrap column GE-Healthcare (5 ml size).
Protein G HiTrap column GE-Healthcare (5 ml size).

The buffer solutions were connected to the buffer's ports of the Biorad LP chromatography system (e.g., binding buffer to inlet C, elution buffer G1 to inlet A, elution buffer G2 to inlet B, elution buffer A1 to inlet D). The Biorad LP chromatography system was turned onto ensure that the buffer cycle is completed and the tubes are connected. The system was run at the maximum capacity with binding buffer to ensure removal of all trapped air bubbles in the tubes. The protein G column was connected to the LP system. The flow rate was adjusted to 2 ml/min and the column was then equilibrated by passing 4-5 volumes of binding buffer (e.g., you need 20-25 mL buffer volume for protein G column of 5 mL size). This was done to ensure the column is prepared for receiving the sample. The samples were diluted 4 times in binding buffer (e.g., 1 mL serum plus 3 mL binding buffer) and then the samples were filtered using 0.45 μm syringe filter. The samples were applied to the column and the unbound proteins were washed by passing 4-5 column volumes of binding buffer (e.g., you need 20-25 mL buffer volume for protein G column of 5 mL size). The solution of the wash-out peak was collected for further purification using protein A columns to purify IgG2 fraction. The bound antibodies were eluted out of the column using elution buffer G1 (elution peak G1 that contains camel IgG3 fraction) by passing 3-4 column volumes (e.g., you need 15-20 mL buffer volume for protein G column of 5 mL size). This step was repeated using elution buffer G2 to elute peak G2 that contains camel IgG1 fraction. The eluted samples were collected in 2 mL size fractions and 100 μL of neutralizing buffer was directly added to each fraction to neutralize the elution buffer acidity. With reference to the purification chart, the fractions containing the elution peaks (G1 & G2) were collected. Each peak fraction was assembled together in one tube and stored at 4° C. for further processing.

The protein G column was re-connected, and the LP tubing system was equilibrated with a binding buffer. Protein A was then connected to the LP system. The collected wash-out peak solution of protein G was applied to the column was applied into the column and then the unbound proteins were washed by passing 4-5 column volumes of binding buffer (e.g., you need 20-25 ml buffer volume for protein A column of 5 ml size). The elution buffer A1 was used to elute the bound antibodies (elution peak A1 that contains camel IgG2 fraction) by passing 3-4 column volumes (e.g., you need 15-20 ml buffer volume for protein A column of 5 ml size). The eluted samples were collected in 2 mL size fractions and 100 Ml of neutralizing buffer was directly added to each fraction to neutralize the elution buffer acidity. With reference to the purification chart, the fractions containing the elution peaks (A1) was collected. Each fraction was assembled together in one tube and stored at 4° C. for further processing.

Each elution peak (G1, G2, A1) was concentrated to a proper volume (according to the expected protein concentration) using a 10 kD or 30 kD Vivispin column by centrifugation at 4000 RPM for 10-15 min.

The protein concentration was measured for each IgG fraction using a Bradford assay pr BCA method. The purity and integrity of each IgG fraction was analyzed using a 12.5% SDS-PAGE. The activity of each IgG fraction was analyzed using an ELISA assay.

Usually the results of the affinity protein purification using Biorad LP chromatography system is expressed as a chart (chromatogram) representing data on the x and y axis. On the y-axis, the absorption unit (A.U) value of the UV lamp detector is represented on the left side as a blue line and the conductivity (mS/cm) value for the conductivity detector is represented on the right side as red line. While the x-axis represents the time or volume of the purification process. When the A.U value increases, this mean that the protein target is going out from the column, while the fluctuation in conductivity value means that the buffer composition and or pH value is changed. There is no need for data calculation for Biorad LP chromatogram.

10. Treatment of Cancer Cell Lines with WNT16B Camelid IgG

Cells were incubated in 96 well plates or 6-well plates 24 hours before the experiment. The cell culture media was replaced by cell culture media containing the WNT16B Camelid IgG1, IgG2,and IgG3 at various concentrations. The WNT16B Camelid IgG antibodies purified from samples before the adult female camel's immunization with the WNT16B protein, along with WNT16B Camelid IgG antibodies purified from samples taken during and after immunization of the adult female camel, and the purified WNT16B camelid IgG from the male calf taken before, during, and after immunization of the mother were each applied to separate plates of media. The WNT16B Camelid IgG concentrations in the cell culture media ranged from 0-10 μg/mL and 10-100 μg/mL. The cells were incubated at 37° C. at 5% CO₂ for 24-72 hours and were collected for an apoptosis analysis at various time points.

11. Apoptosis Analysis of Cancer Cell Lines After Treatment with WNT16B Camelid IgG

An MTT assay was used as per manufacturer's protocol to analyze cell viability after the cells were treated with WNT16B camelid IgG antibodies. Furthermore, an Annexin V FITC Apoptosis detection kit was used according to the manufacturer's protocol and analyzed by flow cytometry to measure levels of apoptosis after the treatment of the cancer cell lines with WNT16B camelid IgG antibodies.

### 12. Transfection for Positive Control

As a precautionary measure, HEK293 cells were transfected with an active WNT16B plasmid DNA. This was carried out in the case the aforementioned cancer cell lines were not producing and secreting a sufficient or significant amount of WNT16B, the transfected HEK293 cells overexpressing WNT16B would be incubated in DMEM media at 37° C. at 5% CO2. The media the transfected HEK293 cells were incubated in (conditioned media) will be taken and used to grow cells that do not produce or secrete active WNT16B. These cells with the conditioned media will then be treated with WNT16B IgG and an apoptosis analysis will be carried out as per methods outlined in Treatment of Cancer Cell Lines with WNT16B Camelid IgG and Apoptosis Analysis of Cancer Cell Lines After Treatment with WNT16B Camelid IgG, respectively.

E. coli bacteria containing an active WNT16B-V5 plasmid (Addgene) was cultured in LB agar plates containing 0.1% of Ampicillin (at a concentration of 100 mg/mL). The bacteria was incubated at 37° C. for 16 hours. A single colony was then selected and grown in 25 mL of LB broth containing 0.1% of Ampicillin (at a concentration of 100 mg/mL) for 16 hours at 37° C. The E. coli bacteria was then lysed and the plasmid DNA was extracted using a Midiprep plasmid DNA isolation kid (QIAGEN). The plasmid DNA containing the active WNT16B-V5 plasmid was stored in 150 μL of 0.1×Tris-EDTA (TE) buffer at 4° C.

A nanodrop analysis was performed in order to measure the concentration of DNA after the plasmid DNA extraction and purification. The concentration of the purified plasmid DNA measured by the nanodrop was 1.098 μg/μL. A restriction digest analysis was also performed in order to ensure that the correct plasmid was extracted and purified from the E. coli cells prior of HEK293 transfection.

Hek293 cells were subcultured into 10 cm plates at a 1:6 subculturing ratio 24 hours before transfection and incubated at 37° C. with 5% CO₂. Next, 4 Eppendorf tubes were labels as follows: Un-transfected 24 hour incubation, Un-transfected 48 hour incubation, Transfected 24 hour incubation, and Transfected 48 hour incubation. To each tube 100 μL of CaCl₂ was added along with 9 μL of the purified plasmid DNA (for total of 10 μg of purified plasmid DNA).

NOTE: FOR THE TUBES LABELED UNTRANSFECTED NO DNA WAS ADDED, AND INSTEAD 9 μL OF 0.1 TE WAS ADDED TO MAKE UP FOR THAT VOLUME.

The tubes were then vortexed for 10 seconds. Next, 391 μL of 0.1×TE was added to the Eppendorf tube to make up the volume to 500 μL. The tube was then vortexed for 10 seconds. Next, 500 μL of 2×HEPES (4-(2-hydroxyethyl)-1-piperazineethanesulfonic acid)) buffer was added to the Eppendorf tube to make a total volume of 1000 μL. The Eppendorf tube was then vortexed for 10 seconds and it was left to stand for 1 minute. The total volume (1000 μL) was taken out of the Eppendorf tube and distributed across the corresponding HEK293 cell plate. The cell culture media was changed for fresh media 6 hours after transfection.

In order to ensure that the transfection of the HEK293 cells was successful, 24 hours after transfection the HEK293 cells on the plates labeled “Untransfected 24 hours” and “Transfected 24 hours” underwent cell lysis and protein extraction as per the procedure outlined in section 2 “Protein Extraction of Cancer Cell Lines” and the lysates were stored at −20° C. After 48 hours following transfection the HEK293 cells on the plates labeled “Untransfected 48 hours” and “Transfected 48 hours” underwent cell lysis and protein extraction as per the procedure outlined in section 2 “Protein Extraction of Cancer Cell Lines” and the lysates were stored at −20° C. The cell lysates collected after transfection underwent a western blot analysis and probed with a V5 monoclonal mouse antibody and imaged using chemiluminescent of horse radish peroxidase conjugated to a secondary anti-mouse antibody in order to determine whether the transfection of the HEK293 cells with the active WNT16B-V5 plasmid DNA was successful.

### RESULTS

### HEK293 Transfection with Active WNT16B-V5 Plasmid DNA

Clear bands were viewed when probed with the V5 antibody through the western blot analysis indicating that the transfection of the HEK293 cells was successful. However, since the cancer cell lines that were being screened for WNT16B expression had a significant and high level of expression and secretion of WNT16B the conditioned media of the transfected HEK293 cells was not needed.

### WNT16B Expression in Cancer Cell Lines

Clear bands were viewed when probed with the rabbit monoclonal WNT16B antibody through the western blot analysis in both cell lysates (protein extraction from cancer cell lines) and the western blot analysis of the cell culture media that the cancer cell lines were grown and incubated in. This indicated that the WNT16B protein was being expressed in the aforementioned cancer cell lines and was being secreted into the media.

### Treatment of Cancer Cell Lines and Apoptosis Analyses

After the aforementioned cancer cell lines were treated and incubated with the WNT16B Camelid IgG antibodies purified from samples during and after immunization of the Dromedary female camel, the apoptosis analyses displayed that more than 50% of cancer cells died within 24-72 hours. While the cancer cell lines treated with the WNT16B Camelid IgG antibodies purified from the calf during and after immunization of the mother Dromedary female camel displayed levels of apoptosis indicating that there was passive immunity after the mother camel was immunization passed down to the calf through the milk. However, the WNT16B camelid IgGs purified from the immunized female camel had a higher titer and specificity for the WNT16B protein than the IgGs purified from the calf. The importance of the passive immunity results indicate that patients could also be treated through consuming milk from camels immunized with the WNT16B protein.

The cells treated with the WNT16B Camelid IgG purified from the samples collected before immunization showed some elevated levels of apoptosis, however the antibodies displayed less specificity than the camelid IgGs purified from the female camel after immunization. This indicated that the naive camelid IgG antibodies can still bind to some degree and block the activity of WNT16B, however the WNT16B IgG antibodies have a much higher targeting and blocking efficiency when blocking the activity of WNT16B when compared to the naive camelid IgG antibodies.

In order to avoid constant immunization of the camel, Lambda phage can be used to produce a phage display library containing the purified WNT16B camelid IgG antibodies. These WNT16B camelid antibodies purified from the immunized camel, calf, and from the phage display libraries could be administered to patients in conjunction with chemotherapy and/or radiotherapy treatments, to block the intercellular signalling activity of WNT16B and to enhance the performance of the chemotherapy and radiotherapy treatments.

The results show that the immunized camel samples of milk, urine, and blood contained specific IgG to block WNT16B activity, and the passive immunity from the mothers milk to the calf displayed that there was a substantial amount of WNT16B specific camelid IgG passed down through the milk of the immunized mother to the calf. As such, this passive immunity can be utilized in cancer treatment by giving the cancer patients milk from the camel immunized with WNT16B in order to produce a passive immunity response. The use of the WNT16B camelid IgG antibodies will enhance the efficiency of the chemotherapy and radiotherapy treatments as it will actively block the WNT16B from taking part in its intercellular signalling pathway activities known to drive cancer cell proliferation and metastasis, as well as cancer recurrence. It will also reduce the chemotherapy and radiotherapy treatment period, and save a lot of lives.

### Expression of Downstream Targets and Changes After Treatment

Since WNT16B intercellular signaling activity was being blocked by the WNT16B camelid IgG antibodies, the amount of β-catenin from the protein extraction lysates after treatment with the antibody were significantly lower, due to β-catenin being degraded by a degradation complex in the cell as a result of WNT16B not being able to bind to the frizzled receptor. The WNT target genes of the WNT-β-catenin signalling pathway that were involved in cancer cell proliferation and cancer cell migration were also downregulated due to the degradation of β-catenin as a result of WNT16B being blocked by the WNT16B camelid IgG antibodies. Thus, WNT16B was unable to bind to the Frizzled transmembrane receptor, leading to the degradation of β-catenin, and therefore not allowing the target genes to be expressed as β-catenin was unable to bind to the Tcf/Lef transcription factors. Therefore, the WNT target genes involved in cancer cell proliferation and migration remained turned off.

### Significance to Cancer Treatment

The benefits of the present disclosure include:
Create new camel industries,
Create new biotechnology using unprecedented camelid IgG in food, medical and pharmaceutical industries,
Improve chemotherapy treatment,
Improve cancer treatment,

### REFERENCES

Binet R. et al. (2009). WNT16B is a new marker of cellular senescence that regulates p53 activity and the phosphoinositide 3-kinase/AKT pathway. Cancer Res 2009; 69:9183-91.
Deffar K., Shi H., et al. (2009) Review: Nanobodies—the new concept in antibody engineering. African Journal of Biotechnology 8, 2645-2652.
Desmyter A, et al. (1996) Crystal structure of a camel single-domain VH antibody fragment in complex with lysozyme. Nature Structural Biology, 3(9), 803-811.
Jassim S. A. A. and Naji M. A. (2001) Review/The desert ship: heritage and science. Biologist (UK) 48 (6), 268-272. http://www.ncbi.nlm.nih.gov/pubmed/11740078
Jassim S. A. A., et al. (2011) WO 2011/104565 A1 Camelid antibodies for use in compositions and methods for the treatment of cancer https://patentscope.wipo.int/search/en/detail.jsf?docld=WO2011104565
Johnson LM, Price DK, Figg WD. (2013) Treatment-induced secretion of WNT16B promotes tumor growth and acquired resistance to chemotherapy: implications for potential use of inhibitors in cancer treatment. Cancer Biol Ther. 14(2):90-91. doi:10.4161/cbt.22636
Martin F, et al. (1997) Affinity selection of a camelied V (H) domain antibody inhibitor of hepatitis C virus NS3 protease. Protein Engineering, 10(5), 607-614.
Meddeb-Mouelhi F, et al. (2003) Immunized camel sera and derived immunoglobulin subclasses neutralizing Androctonus australis hector scorpion toxins. Toxicon 42(7):785-791.
Muyldermans S and Lauwereys M (1999) Unique single-domain antigen binding fragments derived from naturally occurring camel heavy-chain antibodies. Journal of Molecular Recognition, 12, 131-140.
Muyldermans S., et al. (2001) Recognition of antigens by single-domain antibody fragments: the superfluous luxury of paired domains. Trends in Biochemical Sciences, 26(4), 230-235.
Perry JM et al. (2020). Overcoming Wnt-β-catenin dependent anticancer therapy resistance in leukaemia stem cells. Nat Cell Biol 22, 689-700. doi:10.1038/s41556-020-0507-y
Revets H., et al. (2005) Nanobodies as novel agents for cancer therapy. Expert. opin. Biol. Theor. 5: 111-124.
Riechmann L and Muyldermans S (1999) Single domain antibodies: comparison of camel V H and camelied human V H domains. Journal of Immunological Methods, 231 (1-2), 25-38.
Sun Y, Campisi J, Higano C, Beer TM, Porter P, Coleman I, et al. (2012) Treatment-induced damage to the tumor microenvironment promotes prostate cancer therapy resistance through WNT16B. Nat Med. 18(9):1359-1368.
Sun Y et al. (2016). SFRP2 augments WNT16B signaling to promote therapeutic resistance in the damaged tumor microenvironment. Oncogene 35: 4321-4334; doi:10.1038/onc.2015.494
Transue T R, et al. (1998) Camel single-domain antibody inhibits enzyme by mimicking carbohydrate substrate. Proteins, 32, 515-522.
Clevers, H., & Nusse, R. (2012). Wnt/β-Catenin Signaling and Disease. Cell, 149(6), 1192-1205. doi:10.1016/j.cell.2012.05.012
Soule, H.D., Vazquez, J., Long, A., Albert, S., and Brennan, M. (1973). A human cell line from a pleural effusion derived from a breast carcinoma. Journal of the National Cancer Institute, 51(5), 1409-1416.
Sun, Y., Campisi, J., Higano, C., Beer, T. M., Porter, P., Coleman, I., True, L., & Nelson, P. S. (2012). Treatment-induced damage to the tumor microenvironment promotes prostate cancer therapy resistance through WNT16B. Nature medicine, 18(9), 1359-1368. https://doi.org/10.1038/nm.2890

What is claimed is:

1. A method for targeting cancer cells comprising:
immunizing a dromedary camel or llama with a WNT16B molecular to generate anti-WNT16B immune cells producing anti-WNT16B IgG antibodies;
isolating the anti-WNT16B IgG antibodies; and
targeting cancer cells with the anti-WNT16B IgG antibodies.

2. The method as in claim 1, wherein isolating the anti-WNT16B IgG antibodies comprises isolating the IgG antibodies from at least one of camelid milk, urine and blood.

3. The method as in claim 1, wherein isolating the anti-WNT16B IgG antibodies comprises:
selecting a set of anti-WNT16B IgG antibodies from the camel or llama immune cells;
cloning the set of anti-WNT16B IgG antibodies in an expression system; and
selecting blocking anti-WNT16B IgG antibodies from the clones.

4. The method as in claim 1, wherein targeting cancer cells comprises providing the anti-WNT16B IgG antibodies and a chemotherapy treatment.

5. The method as in claim 1, wherein targeting cancer cells comprises providing the anti-WNT16B IgG antibodies and a radiation therapy.

6. The method as in claim 1, wherein isolating the anti-WNT16B IgG antibodies and targeting cancer cells comprises:
collecting milk from the camel or llama; and
providing the milk to a patient for targeting the cancer cells of the patient by passive immunity.

7. A method to detect cancer cells comprising:
immunizing a dromedary camel or llama with a WNT16B molecule to generate anti-WNT16B immune cells;
selecting a set of anti-WNT16B IgG antibodies from the camel or llama immune cells;
cloning the set of anti-WNT16B IgG antibodies in an expression system;
selecting anti-WNT16B IgG antibodies from the clones; and
detecting cancer cells with the anti-WNT16B IgG antibodies.

8. The method as in claim 7, wherein detecting cancer cells with the anti-WNT16B IgG antibodies comprises: detecting cancer cells in vitro in a histology sample.

9. The method as in claim 7, wherein detecting cancer cells with the anti-WNT16B IgG antibodies comprises detecting cancer cells in vivo.
